(19) United States
(12) Patent Application Publication
(10) Pub. No.: US 2006/0094110 A1
(43) Pub. Date: May 4, 2006

(54) Use of spliceosome mediated RNA trans-splicing for immunotherapy

(76) Inventors: Gerard J. McGarrity, Gaithersburgh, MD (US); Mariano A. Garcia-Blanco, Durham, NC (US)

Correspondence Address: ARENT FOX PLLC, 1675 BROADWAY, NEW YORK, NY, 10019, US

(21) Appl. No.: 11/193,682

(22) Filed: Jul. 29, 2005

Related U.S. Application Data
(60) Provisional application No. 60/592,607, filed on Jul. 30, 2004.

(51) Int. Cl. C07H 21/02; C07H 21/04

(52) U.S. Cl. 435/325; 536/23.1

(57) ABSTRACT

Methods and compositions for generating novel nucleic acid molecules through targeted spliceosomal mediated trans-splicing that result in expression of an immunogenic polypeptide. The invention includes pre-trans-splicing molecules (PTMs) designed to interact with a target precursor messenger RNA molecule (target pre-mRNA) and mediate a trans-splicing reaction resulting in the generation of a novel chimeric RNA molecule (chimeric RNA) capable of encoding the immunogenic polypeptide, recombinant vector systems capable of expressing the PTMs of the invention, and cells expressing said PTMs. The target pre-mRNA are those encoding proteins that function in antigen uptake, antigen presentation and chaperoning. The methods of the invention encompass contacting the PTMs of the invention with a target pre-mRNA, under conditions in which a portion of the PTM is trans-spliced to a portion of the target pre-mRNA to form a chimeric mRNA molecule capable of encoding an immunogenic polypeptide.

The present application claims benefit under 35 U.S.C. § 119 to provisional application No. 60/592,607 filed on Jul. 30, 2004.

### 1. INTRODUCTION

The present invention provides methods and compositions for generating novel nucleic acid molecules through targeted spliceosomal mediated trans-splicing that result in expression of an antigenic polypeptide. The compositions of the invention include pre-trans-splicing molecules (PTMs) designed to interact with a target precursor messenger RNA molecule (target pre-mRNA) and mediate a trans-splicing reaction resulting in the generation of a novel chimeric RNA molecule (chimeric RNA) capable of encoding an antigenic polypeptide. The invention also provides recombinant vector systems capable of expressing the PTMs of the invention, and cells expressing said PTMs. The target pre-mRNAs are those encoding proteins that function in antigen uptake, presentation or chaperoning. The methods of the invention encompass contacting the PTMs of the invention with a target pre-mRNA, under conditions in which a portion of the PTM is trans-spliced to a portion of the target pre-mRNA to form a chimeric mRNA molecule capable of encoding an immunogenic polypeptide. The methods and compositions of the present invention can be used to induce an immune response against a variety of different polypeptides. Such polypeptides include, but are not limited to, those encoded by infectious agents, such as viral, bacterial, fungal or parasitic agents. Additionally, the immunogenic polypeptides may be tumor-specific and/or tumor-associated antigens and/or antigens associated with an autoimmune or other disease. These polypeptides include not only tumor-specific antigens but tissue-specific self-antigens, as well as self antigens involved with autoimmune disease. The tissue specific self-antigens provide a means for inducing an autoimmune reaction to attack tumors. The compositions and methods of invention provide a novel means for vaccination against immunogenic polypeptides.

### 2. BACKGROUND OF THE INVENTION

### 2.1. RNA Splicing

DNA sequences in the chromosome are transcribed into pre-mRNAs which contain coding regions (exons) and generally also contain intervening non-coding regions (introns). Introns are removed from pre-mRNAs in a precise process called splicing (Chow et al., 1977, Cell 12:1-8; and Berget, S. M. et al., 1977, Proc. Natl. Acad. Sci. USA 74:3171-3175). Splicing takes place as a coordinated interaction of several small nuclear ribonucleoprotein particles (snRNP's) and many protein factors that assemble to form an enzymatic complex known as the spliceosome (Moore et al., 1993, in The RNA World, R. F. Gestland and J. F. Atkins eds. (Cold Spring Harbor Laboratory Press, Cold Spring Harbor, N.Y.); Kramer, 1996, Annu. Rev. Biochem., 65:367-404; Staley and Guthrie, 1998, Cell 92:315-326).

In most cases, the splicing reaction occurs within the same pre-mRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Nat'l. Acad. Sci. USA, 87:8879; Davis et al., 1995, J. Biol. Chem. 270:21813) and in plant mitochondria (Malek et al., 1997, Proc. Nat'l. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of splice leader trans-splicing, which is nearly identical to that of conventional cis-splicing, proceeds via two phosphoryl transfer reactions. The first causes the formation of a 2′-5′ phosphodiester bond producing a ‘Y’ shaped branched intermediate, equivalent to the lariat intermediate in cis-splicing. The second reaction, exon ligation, proceeds as in conventional cis-splicing. In addition, sequences at the 3′ splice site and some of the snRNPs which catalyze the trans-splicing reaction, closely resemble their counterparts involved in cis-splicing.

Trans-splicing may also refer to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Nat'l. Acad. Sci. USA 86:8020). In addition, trans-splicing of c-myb pre-RNA has been demonstrated (Vellard, M. et al. Proc. Nat'l. Acad. Sci., 1992 89:2511-2515) and more recently, RNA transcripts from cloned SV40 trans-spliced to each other were detected in cultured cells and nuclear extracts (Eul et al., 1995, EMBO. J. 14:3226). However, naturally occurring trans-splicing of mammalian pre-mRNAs is thought to be a rare event (Flouriot G. et al., 2002 J. Biol. Chem: Finta, C. et al., 2002 J. Biol Chem 277:5882-5890).

In vitro trans-splicing has been used as a model system to examine the mechanism of splicing by several groups (Konarska & Sharp, 1985, Cell 46:165-171 Solnick, 1985, Cell 42:157; Chiara & Reed, 1995, Nature 375:510; Pasman and Garcia-Blanco, 1996, Nucleic Acids Res. 24:1638). Reasonably efficient trans-splicing (30% of cis-spliced analog) was achieved between RNAs capable of base pairing to each other, splicing of RNAs not tethered by base pairing was further diminished by a factor of 10. Other in vitro trans-splicing reactions not requiring obvious RNA-RNA interactions among the substrates were observed by Chiara & Reed (1995, Nature 375:510), Bruzik J. P. & Maniatis, T. (1992, Nature 360:692) and Bruzik J. P. and Maniatis, T., (1995, Proc. Nat'l. Acad. Sci. USA 92:7056-7059). These reactions occur at relatively low frequencies and require specialized elements, such as a downstream 5′ splice site or exonic splicing enhancers.

In addition to splicing mechanisms involving the binding of multiple proteins to the precursor mRNA which then act to correctly cut and join RNA, a third mechanism involves cutting and joining of the RNA by the intron itself, by what are termed catalytic RNA molecules or ribozymes. The cleavage activity of ribozymes has been targeted to specific RNAs by engineering a discrete “hybridization” region into the ribozyme. Upon hybridization to the target RNA, the catalytic region of the ribozyme cleaves the target. It has been suggested that such ribozyme activity would be useful for the inactivation or cleavage of target RNA in vivo, such as for the treatment of human diseases characterized by production of foreign of aberrant RNA. In such instances small RNA molecules are designed to hybridize to the target RNA and by binding to the target RNA prevent translation of the target RNA or cause destruction of the RNA through activation of nucleases. The use of antisense RNA has also been proposed as an alternative mechanism for targeting and destruction of specific RNAs.

Using the Tetrahymena group I ribozyme, targeted trans-splicing was demonstrated in E. coli. (Sullenger B. A. and Cech. T. R., 1994, Nature 341:619-622), in mouse fibroblasts (Jones, J. T. et al., 1996, Nature Medicine 2:643-648), human fibroblasts (Phylacton, L. A. et al. Nature Genetics 18:378-381) and human erythroid precursors (Lan et al., 1998, Science 280:1593-1596). For a review of clinically relevant technologies to modify RNA see Sullenger and Gilboa, 2002 Nature 418:252-8. The present invention relates to the use of targeted trans-splicing mediated by native mammalian splicing machinery, i.e., spliceosomes, to reprogram or alter the coding sequence of a targeted m-RNA.

U.S. Pat. Nos. 6,083,702, 6,013,487 and 6,280,978 describe the use of PTMs to mediate a trans-splicing reaction by contacting a target precursor mRNA to generate novel chimeric RNAs.

### 2.2. Immunotherapy

Antigen processing and presentation are processes that occur within a cell that result in proteolysis of proteins and association of the protein fragments with MHC molecules. The peptide-MHC molecules are then expressed at the cell surface where they are recognized by the T cell receptor. This process is important for induction of a protective immune response against the antigen.

The path leading to the association of protein fragments with MHC molecules differs depending on whether class I or class II MHC molecules are involved. MHC I molecules present self- or pathogen derived antigens that are synthesized within the cell, whereas exogenous antigens derived via endocytic uptake are loaded onto MHC II molecules for presentation to CD4+ T cells. Some antigen presenting cells are also specialized to process exogenous antigens into the MHC I pathway for presentation to CD8+ T cells, a process known as cross-presentation.

A variety of different approaches have been used to induce immunity against pathogens and cells. Standard vaccine technologies have utilized living attenuated infectious organisms or inactivated virulent forms to induce a humoral response against such infectious organisms. Alternatively, induction of immunity has included the use of polypeptides that confer protection against that polypeptide as well as the infectious organism that expresses that polypeptide.

In a more recent development, nucleic acids encoding for polypeptides of the infectious organism have been used as vaccinating agents. While these types of experiments have been successful in animals, clinical studies, in humans have, for the most part, been disappointing (see, Maloy et al., PNAS 98:3299-3303). Typically, higher doses of nucleic acids have been required in the vaccines to induce measurable immunological responses, and overall, results have been disappointing.

Attempts have also been made to induce immunity to antigens that are either tumor specific or tumor associated with the objective of inducing an immunologic response to cancer, resulting either in prophylactic protection against tumor challenge, or as a therapeutic vaccine. As observed in nucleic acid vaccines for pathogenic organisms, significant success has been achieved in animal models, but again, results in humans have been disappointing.

However, when murine homologues of melanoctye-specific differentiation antigens targeted by T cells from human melanoma patients were assayed for their ability to induce a tissue-specific autoimmunity and to offer protection against challenge by melanoma cells, it was found that TRP-1 induced vitiligo in mice and protected against challenge by C57BL6 derived B16 melanoma (Overwijk et al. PNAS 96:2982-2987, 1999).

Attempts to improve the effectiveness of nucleic acid vaccination have included concomitant approaches to improve the cellular machinery of antigen uptake, antigen presentation and even prolonging the duration of antigen processing, as well as the lifetime of antigen presenting cells. For example, the duration of antigen presentation in dentritic cells could be prolonged through the use of a peptide derived from tyrosinase-related protein 2 (TRP-2) covalently linked to a cell penetrating peptide (CPP) (Wnad et al., 2002, Nature Biotechnology, 20:149-154). Enhanced nucleic acid vaccine potency was also observed by co-administering nucleic acid molecules capable of encoding anti-apoptotic proteins. The use of such a method was found to prolong the survival of dendritic cells and, presumably, improved the immune response. Of the anti-apoptotic agents studied, BCL-XL seems to have the greatest effect in generation of antigen-specific immune responses and antitumor effects (Kim et al., J. Clinical Invest., 2003 112:109-117).

While most nucleic acid vaccines have been administered intramuscularly or intradermally, it has been demonstrated that intralymphatic immunization of a plasmid vaccine directly into lymphoid tissues generated an immune response that was 100 to 1,000 fold more efficient than other routes of administration (Maloy et al., Proc. Natl. Acad. Sci., 2001 98:3299-3303). Further, to target the antigen to the endosomal and lysosomal compartments, a chimeric gene encoding a model antigen of human papillomavirus (HPV) 16E7 (HPV-1 6E7) was fused to the transmembrane and cytoplasmic region of LAMP-1 (Wu et al., Proc. Natl. Acad. Sci., 1995, 92:11671-11675). Results showed that this strategy enhanced MHC II presentation and increased vaccine potency in vivo.

As reported by Stevanovic (Transplant Immunology 14:171-174, 2005), MHC Class I antigen processing features three major steps: antigen processing by the proteosome, transport by TAP and binding to nascent MHC molecules. The present invention allows trans-splicing into antigen processing molecules such as TAP or other transcripts that play a key role in antigen presentation or processing so that administered antigens can be processed and presented using the same pathways and cells compartments as occur naturally.

### 3. SUMMARY OF THE INVENTION

The present invention relates to compositions and methods for generating novel nucleic acid molecules through spliceosome-mediated targeted trans-splicing that are capable of encoding polypeptides for inducing an immune response. In one particular embodiment, the immune response can be a humoral or cellular response. The compositions and methods of the invention can be used prophylactically or therapeutically. The methods and compositions of the invention may be used in vaccinations to stimulate a protective immune response against said polypeptide. Such polypeptides include those encoded by pathogenic organisms such as, for example, viruses, fungi, bacteria and parasites. The methods and compositions of the invention may also be used to eliminate cells of the subject that express tumor-specific, as well as tissue specific self-antigens, including tumor cells. The compositions of the invention include pre-trans-splicing molecules (hereinafter referred to as “PTMs”) designed to interact with a natural target pre-mRNA molecule (hereinafter referred to as “pre-mRNA”) and mediate a spliceosomal trans-splicing reaction resulting in the generation of a novel chimeric RNA molecule (hereinafter referred to as “chimeric RNA”). The chimeric RNA is designed to encode a fusion protein comprising a polypeptide of interest fused to a polypeptide involved in antigen uptake, presentation or chaperoning. Such fusion proteins are particularly well suited for induction of a protective immune response against said fusion protein. The methods of the invention encompass contacting the PTMs of the invention with a natural target pre-mRNA, that normally involves proteins involved in antigenic uptake, presentation or chaperoning, under conditions in which a portion of the PTM is spliced to the natural pre-mRNA to form a novel chimeric RNA. The PTMs of the invention are genetically engineered so that the novel chimeric RNA resulting from the trans-splicing reaction may encode an antigenic polypeptide that provides beneficial health benefits. The resulting chimeric RNA can be monocistronic, bi-cistronic or polycistronic. Bi-cistronic RNAs could be produced through the use of sequences such as internal ribozyme entry site (IRES) or the use of, for example, A2 or similar sequences from foot and mouth disease virus that allows cleavage of the message at the 3′ end of a sequences but still allowing translation to continue.

In particular, the compositions of the invention include pre-trans-splicing molecules (hereinafter referred to as “PTMs”) designed to interact with a pre-mRNA molecule (hereinafter referred to as “target pre-mRNA”) encoding for proteins that function in antigen uptake, presentation or chaperoning and mediate a spliceosomal trans-splicing reaction resulting in the generation of a novel chimeric RNA molecule (hereinafter referred to as “chimeric RNA”).

The compositions of the invention include PTMs designed to interact with a target pre-mRNA and mediate a spliceosomal trans-splicing reaction resulting in the generation of a novel chimeric RNA molecule. Such PTMs are designed to produce an immunogenic polypeptide, which is capable of stimulating a protective immune response. The general design, construction and genetic engineering of PTMs and demonstration of their ability to successful mediate trans-splicing reactions within the cell are described in detail in U.S. Pat. Nos. 6,083,702, 6,013,487 and 6,280,978 as well as patent Ser. Nos. 09/756,095, 09/756,096, 09/756,097 and 09/941,492, the disclosures of which are incorporated by reference in their entirety herein.

The methods of the invention encompass contacting the PTMs of the invention with a target pre-mRNA, under conditions in which a portion of the PTM is spliced to the target pre-mRNA to form a novel chimeric RNA. The methods of the invention comprise contacting the PTMs of the invention with a cell expressing a target pre-mRNA, under conditions in which the PTM is taken up by the cell and a portion of the PTM is trans-spliced to a portion of the target pre-mRNA to form a novel chimeric RNA molecule that results in expression of an antigenic polypeptide. Alternatively, nucleic acid molecules encoding the PTMs of the invention may be delivered into a target cell followed by expression of the nucleic acid molecule to form a PTM capable of mediating a trans-splicing reaction. The PTMs of the invention are genetically engineered so that the novel chimeric RNA resulting from the trans-splicing reaction encodes an antigenic polypeptide which is capable of stimulating an immune response. Thus, the methods and compositions of the invention can be used to immunize against diseases arising from infection by a variety of different pathogenic microorganisms, including but not limited to viral, bacterial, fungal and parasitic infections. Additionally, the methods and compositions can be used to treat a variety of different types of cancers through stimulation of an immune response against tumor-specific and/or tumor-associated antigens, including tissue-specific self-antigens.

The present invention also provides a means to investigate which target transcript is most appropriate for processing, chaperoning and presenting a particular antigen. For example, Andersson and Barry (Mol. Ther. 10:432-438, 2004) showed that the utility of an antigen-ubiquitin fusion is due to the targeting of ubiquitin to the proteosome. However, as these authors point out, while ubiquitin fusion enhances CD8⁺ responses, each construct must be optimized for maximal response. While the use of ubiquitin fusions have worked in some systems (Leachman et al. J. Virol. 76:7616-7624, 2002), they have not been universally successful (Heng et al. Cell Immun 215:20-31, 2002).

In a further embodiment, a PTM may be used that encodes an immunoregulatory molecule such as LAMP-1, ubiquitin, heat shock proteins, for example. This type of PTM is characterized by the lack of a specific binding domain, meaning that it will trans-splice promiscuously to many transcripts. This immuno-PTM is introduced into a cell that is, for example, a cancer cell expressing a tumor associated protein or a cell infected with a particular virus or other microorganism. The immuno-PTM will trans-splice randomly. Following trans-splicing, an immuno-OTM—antigen cDNA expression library is prepared. This library may be used for immunization since the tumor-specific or viral-specific antigens will be better immunogens. The patient's lymphocytes could be used with in vitro selection methods.

### 4. BRIEF DESCRIPTION OF THE DRAWINGS

FIG. 1. Schematic representation of trans-splicing using a PTM with no specific binding domain which will trans-splice randomly to all transcripts, including involved in antigen processing, chaperoning and presentation. Use of a molecular library approach can readily select the most optimized target for each antigen.

FIG. 2. shows a tool for genomic annotation of transcripts in antigen processing.

FIG. 3. Schematic representation of trans-splicing to a representative antigen processing transcript, LAMP-1. The antigen of interest is trans-spliced into antigen processing/presentation transcript to allow natural processing to MHC I and/or MHC II molecules. LAMP-1 pre-mRNA is ˜27 kb which is trans-spliced to create either a fusion protein or two separate proteins using IRES or A2.

FIG. 4. Schematic representation of different trans-splicing reactions. (a) trans-splicing reactions between the target 5‘splice site and PTM’s 3′ splice site, (b) trans-splicing reactions between the target 3‘splice site and PTM’s 5′ splice site and (c) replacement of an internal exon by a double trans-splicing reaction in which the PTM carries both 3′ and 5′ splice sites. BD, binding domain; BP, branch point sequence; PPT, polypyrimidine tract; and ss, splice sites.

### DETAILED DESCRIPTION OF THE INVENTION

The present invention relates to novel compositions comprising pre-trans-splicing molecules (PTMs) and the use of such molecules for generating novel nucleic acid molecules. The PTMs of the invention comprise (i) one or more target binding domains that are designed to specifically bind to a target pre-mRNA wherein said target pre-mRNA normally involves a protein involved in antigen uptake, presentation or chaperoning, (ii) a 3′ splice region that includes a branch point, pyrimidine tract and a 3′ splice acceptor site and/or a 5′ splice donor site; and (iii) additional nucleotide sequences such as those encoding an antigenic polypeptide of interest. The PTMs of the invention may further comprise one or more spacer regions that separate the RNA splice site from the target binding domain.

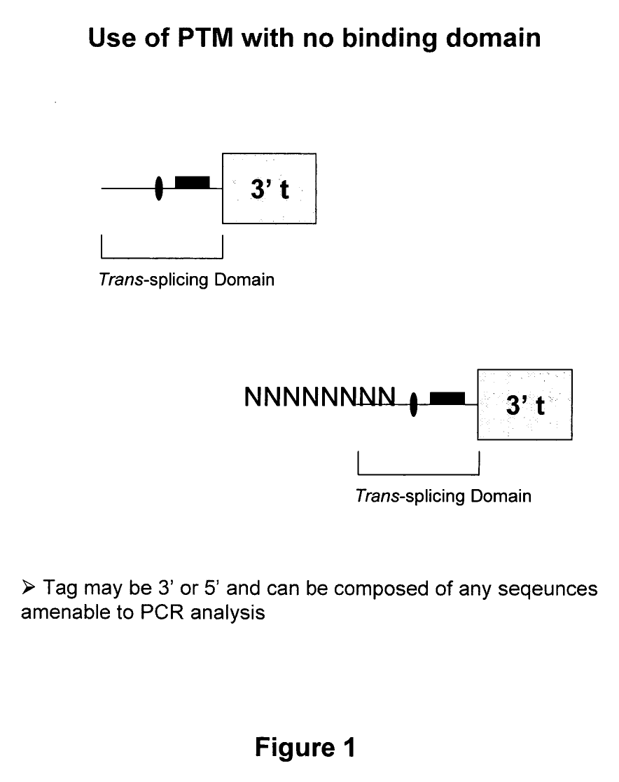

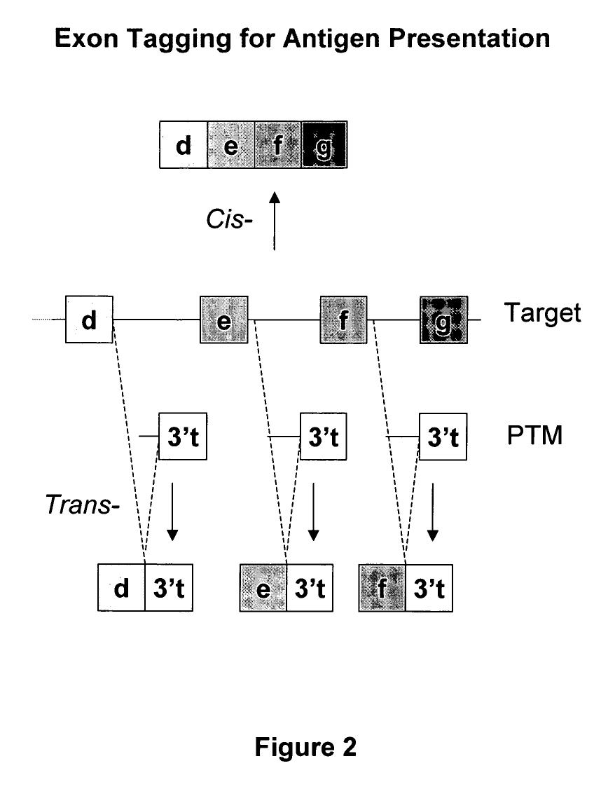

In a variation, the PTM could be without any specific binding domain, meaning that it will trans-splice randomly in any cell it enters, producing a collection of chimeric RNAs, as shown in FIGS. 1 and 2. Use of an immunoPTM-antigen cDNA expression library can select the most optimal configuration for antigen processing, chaperoning and presentation. Such non-specific PTMs will randomly trans-splice to the vast majority of transcripts and exons. For example, PTMs encoding luciferase and lacking a specific binding domain have been trans-spliced into 86% of albumin exons, and 100% of exons in the following human transcripts: alpha 1-antitrypsin, insulin like growth factor and fibroblast growth factor receptor, showing the high efficiency in these transcripts of high and medium abundance. This is an effective model system for trans-splicing antigens to a diverse set of transcripts that process, chaperone or present antigens.

The methods of the invention encompass contacting the PTMs of the invention with a target pre-mRNA under conditions in which a portion of the PTM is trans-spliced to a portion of the target pre-mRNA to form a novel chimeric RNA that results in expression of an antigenic polypeptide of interest against.

### 5.1. Structure of the Pre-Trans-Splicing Molecules

The present invention provides compositions for use in generating novel chimeric nucleic acid molecules capable of encoding an antigenic polypeptide of interest through targeted trans-splicing. The PTMs of the invention comprise (i) one or more target binding domains that targets binding of the PTM to a target pre-mRNA (ii) a 3′ splice region that includes a branch point, pyrimidine tract and a 3′ splice acceptor site and/or 5′ splice donor site; and (iii) sequences encoding an antigenic polypeptide of interest. The PTMs of the invention may also include at least one of the following features: (a) binding domains targeted to intron sequences in close proximity to the 3′ or 5′ splice signals of the target intron, (b) mini introns, (c) ISAR (intronic splicing activator and repressor) consensus binding sites, (d) ribozyme sequences and/or (e) safety sequences. The PTMs of the invention may further comprise one or more spacer regions to separate the RNA splice site from the target binding domain.

The general design, construction and genetic engineering of such PTMs and demonstration of their ability to mediate successful trans-splicing reactions within the cell are described in detail in U.S. Pat. Nos. 6,083,702, 6,013,487 and 6,280,978 as well as patent Ser. Nos. 09/941,492, 09/756,095, 09/756,096 and 09/756,097 the disclosures of which are incorporated by reference in their entirety herein.

The target binding domain of the PTM endows the PTM with a binding affinity for the target pre-mRNA, in this case, a RNA encoding a protein that functions in antigen uptake, presentation or chaperoning. Proteins that function in antigen uptake, include but are not limited to TAP-1, TAP-2 and CD44. Proteins that function in antigen presentation include, but are not limited to LAMP-1, LAMP-3, HLA-DNA, FH3, B7-DC OR B7-1. Proteins that function in antigen chaperoning include, but are not limited to heat shock proteins, such as heat shock protein (HSP) 60 (Hsp60), Hsp70, Hsp 96 and gp 96.

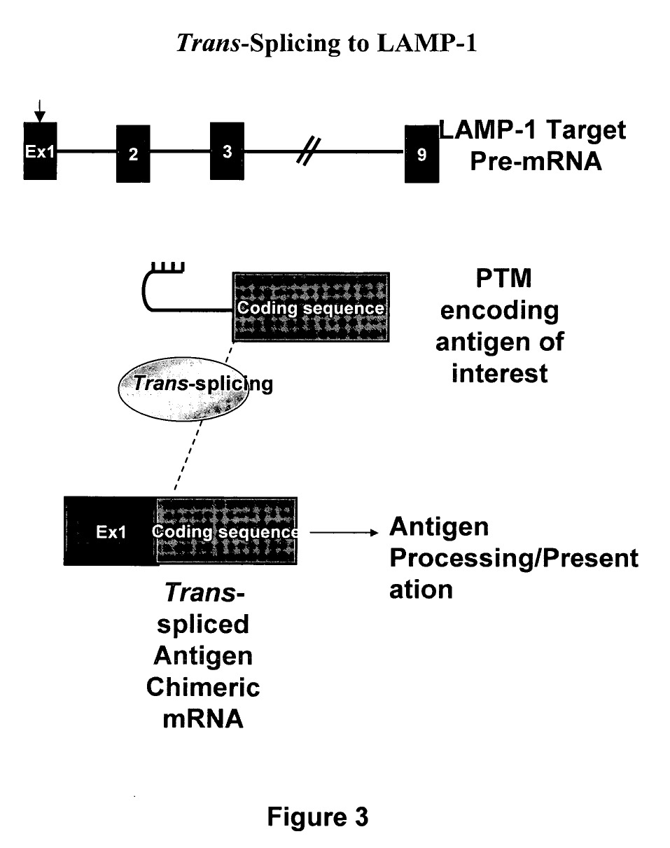

In an exemplary embodiment of the invention, PTMs may be designed to target the LAMP-1 pre-RNA, as shown in FIG. 3. The human LAMP-1 gene is a 25 kb gene consisting of 9 exons; the mRNA is 2.5 kb. The first exon encodes a signal peptide and the last exon contains a long stretch of untranscribed sequences. The intracellular targeting of LAMP-1 is controlled by the cytoplasmic domain Tyr-Glu-Thr-Ile at the C′ terminus of the cytoplasmic domain (Guarnieri, F. G. et al. J. Biol. Chem. 268:1941-1946, 1993). The motif Tyr-X-X-hydrophobic residues mediates lysosomal membrane targeting of lysosome-associated membrane protein 1 in both mouse and human. In a specific embodiment of the invention, a PTM is produced that mediates a trans-splicing reaction into intron 1 of the LAMP-1 target RNA. In another embodiment of the invention, a PTM is produced that mediates a trans-splicing reaction into intron 8 of the LAMP-1 gene. For example, in a specific embodiment of the invention, processing may be improved by placing the nucleic acid encoding the antigenic peptide of interest at the 3′ end by trans-splicing exon 9, followed by a poly A signal, at the end of the nucleic acid encoding the antigenic peptide of interest.

In another embodiment, a PTM lacking a specific binding domain and encoding the antigen of interest could be utilized. This can be used to trans-splice to many targets, especially in cells of the immunological series in which genes for antigen processing, chaperoning or presentation are transcribed. Through the use of cDNA expression libraries and rapid amplification of cDNA ends (RACE), the transcripts that are associated with processing, chaperoning and presentation of any specific antigen can be identified.

As used herein, a target binding domain is defined as any molecule, i.e., nucleotide, protein, chemical compound, etc., that confers specificity of binding and anchors the pre-mRNA closely in space to the PTM so that the spliceosome processing machinery of the nucleus can trans-splice a portion of the PTM to a portion of the pre-mRNA.

The target binding domain of the PTM may contain multiple binding domains which are complementary to and in anti-sense orientation to the targeted region of the selected pre-mRNA. The target binding domains may comprise up to several thousand nucleotides. In preferred embodiments of the invention the binding domains may comprise at least 10 to 30 and up to several hundred or more nucleotides. The specificity of the PTM may be increased significantly by increasing the length of the target binding domain. For example, the target binding domain may comprise several hundred nucleotides or more. In addition, although the target binding domain may be “linear” it is understood that the RNA will very likely fold to form secondary structures that may stabilize the complex thereby increasing the efficiency of splicing. A second target binding region may be placed at the 3′ end of the molecule and can be incorporated into the PTM of the invention. Absolute complementarity, although preferred, is not required. A sequence “complementary” to a portion of an RNA, as referred to herein, means a sequence having sufficient complementarity to be able to hybridize with the target pre-mRNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch or length of duplex by use of standard procedures to determine the stability of the hybridized complex.

Binding may also be achieved through other mechanisms, for example, through triple helix formation, aptamer interactions, antibody interactions or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

In a specific embodiment of the invention, the target binding domain is complementary and in anti-sense orientation to sequences in close proximity to the region of the target pre-mRNA targeted for trans-splicing. For example, a target binding domain may be defined as any molecule, i.e., nucleotide, protein, chemical compound, etc., that confers specificity of binding and anchors the pre-mRNA closely in space to the PTM so that the spliceosome processing machinery of the nucleus can trans-splice a portion of the PTM to a portion of the target, pre-mRNA.

The PTM molecule also contains a 3′ splice region that includes a branchpoint sequence and a 3′ splice acceptor AG site and/or a 5′ splice donor site. The 3′ splice region may further comprise a polypyrimidine tract. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and /=the splice site). The 3′ splice site consists of three separate sequence elements: the branchpoint or branch site, a polypyrimidine tract and the 3′ consensus sequence (YAG). The branch point consensus sequence in mammals is YNYURAC (Y=pyrimidine; N=any nucleotide). The underlined A is the site of branch formation. A polypyrimidine tract is located between the branch point and the splice site acceptor and is important for different branch point utilization and 3′ splice site recognition. Recently, pre-mRNA introns beginning with the dinucleotide AU and ending with the dinucleotide AC have been identified and referred to as U12 introns. U12 intron sequences as well as any sequences that function as splice acceptor/donor sequences may also be used to generate the PTMs of the invention.

A spacer region to separate the RNA splice site from the target binding domain may also be included in the PTM. The spacer region may be designed to include features such as (i) stop codons which would function to block translation of any unspliced PTM and/or (ii) sequences that enhance trans-splicing to the target pre-mRNA.

In a preferred embodiment of the invention, a “safety” is also incorporated into the spacer, binding domain, or elsewhere in the PTM to prevent non-specific trans-splicing. This is a region of the PTM that covers elements of the 3′ and/or 5′ splice site of the PTM by relatively weak complementarity, preventing non-specific trans-splicing. The PTM is designed in such a way that upon hybridization of the binding/targeting portion(s) of the PTM, the 3′ and/or 5′splice site is uncovered and becomes fully active.

Such “safety” sequences comprises one or more complementary stretches of cis-sequence (or could be a second, separate, strand of nucleic acid) which binds to one or both sides of the PTM branch point, pyrimidine tract, 3′ splice site and/or 5′ splice site (splicing elements), or could bind to parts of the splicing elements themselves. This “safety” binding prevents the splicing elements from being active (i.e. block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

A nucleotide sequence encoding the antigenic polypeptide of interest, i.e., the polypeptide against which induction of an immune response is desired, is also included in the PTM of the invention. For example, nucleotide sequences encoding gene products associated with infection with pathogenic microorganisms such as, for example, viruses, bacteria, fungi and parasites may be included in the PTMS. Additionally, antigenic polypeptides of interest may include tumor-specific or tumor-associated antigens such as, for example, Her2/Neu, CEA, MUC1, TRP-1, TRP-2 and MARTI/MelanA.

Antigenic polypeptides may also include tissue-specific self-antigens. For example, known antigen or epitope mimicry between antigens on infectious organisms and self-antigens may be used to design antigenic polypeptides. In a specific embodiment of the invention, polypeptides associated with autoimmune disease such as, for example, between the spirochete etiologic agent of Lyme disease and LFA-1 may be utilized to induce a protective immune response. Antigenic polypeptides may also include tissue-specific self-antigens associated with tumor antigens. Such antigenic polypeptides may be used in cancer therapy.

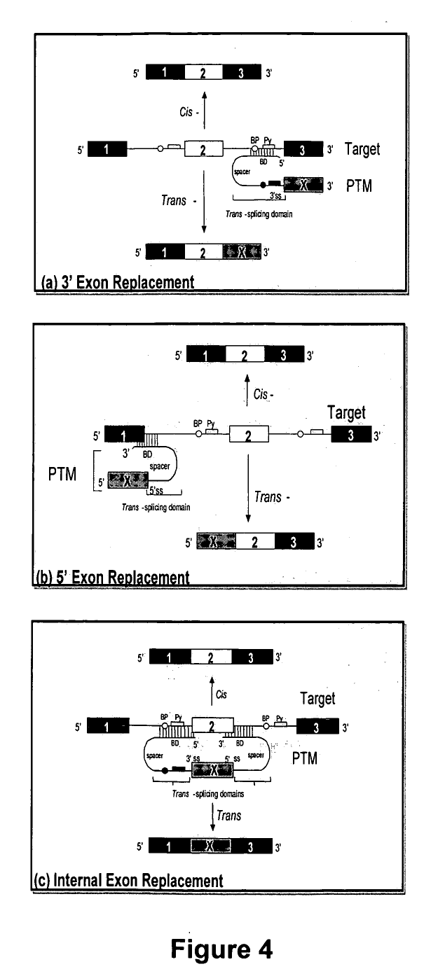

The PTM's of the invention may be engineered to contain a single exon sequence, multiple exon sequences, or alternatively the complete set of exon sequences encoding the antigen of interest. The number and identity of the sequences to be used in the PTMs will depend on the type of trans-splicing reaction, i.e., 5′ exon replacement, 3′ exon replacement or internal exon replacement that will occur (see, FIG. 4).

The present invention further provides PTM molecules wherein the coding region of the PTM is engineered to contain mini-introns. The insertion of mini-introns into the coding sequence of the PTM is designed to increase definition of the exon and enhance recognition of the PTM donor site. Mini-intron sequences to be inserted into the coding regions of the PTM include small naturally occurring introns or, alternatively, any intron sequences, including synthetic mini-introns, which include 5′ consensus donor sites and 3′ consensus sequences which include a branch point, a 3′ splice site and in some instances a pyrimidine tract.

The mini-intron sequences are preferably between about 60-150 nucleotides in length, however, mini-intron sequences of increased lengths may also be used. In a preferred embodiment of the invention, the mini-intron comprises the 5′ and 3′ end of an endogenous intron. In preferred embodiments of the invention the 5′ intron fragment is about 20 nucleotides in length and the 3′ end is about 40 nucleotides in length.

In a specific embodiment of the invention, an intron of 528 nucleotides comprising the following sequences may be utilized. Sequence of the intron construct is as follows:

5′ fragment sequence:
Gtagttcttttgttcttcactattaagaacttaatttggtgtccatgtct
ctttttttttctagtttgtagtgctggaaggtatttttggagaaattctt
acatgagcattaggagaatgtatgggtgtagtgtcttgtataatagaaat
tgttccactgataatttactctagttttttatttcctcatattattttca
gtggctttttcttccacatctttatattttgcaccacattcaacactgta
gcggccgc.
3′ fragment sequence:
Ccaactatctgaatcatgtgccccttctctgtgaacctctatcataatac
ttgtcacactgtattgtaattgtctcttttactttcccttgtatcttttg
tgcatagcagagtacctgaaacaggaagtattttaaatattttgaatcaa
atgagttaatagaatctttacaaataagaatatacacttctgcttaggat
gataattggaggcaagtgaatcctgagcgtgatttgataatgacctaata
atgatgggttttatttccag

In yet another specific embodiment of the invention, consensus ISAR sequences are included in the PTMs of the invention (Jones et al., NAR 29:3557-3565). Proteins bind to the ISAR splicing activator and repressor consensus sequence which includes a uridine-rich region that is required for 5′ splice site recognition by U1 SnRNP. The 18 nucleotide ISAR consensus sequence comprises the following sequence: GGGCUGAUUUUUCCAUGU. When inserted into the PTMs of the invention, the ISAR consensus sequences are inserted into the structure of the PTM in close proximity to the 5′ donor site of intron sequences. In an embodiment of the invention the ISAR sequences are inserted within 100 nucleotides from the 5′ donor site. In a preferred embodiment of the invention the ISAR sequences are inserted within 50 nucleotides from the 5′ donor site. In a more preferred embodiment of the invention the ISAR sequences are inserted within 20 nucleotides of the 5′ donor site.

The compositions of the invention further comprise PTMs that have been engineered to include cis-acting ribozyme sequences. The inclusion of such sequences is designed to reduce PTM translation in the absence of trans-splicing or to produce a PTM with a specific length or defined end(s). The ribozyme sequences that may be inserted into the PTMs include any sequences that are capable of mediating a cis-acting (self-cleaving) RNA splicing reaction. Such ribozymes include but are not limited to hammerhead, hairpin and hepatitis delta virus ribozymes (see, Chow et al. 1994, J Biol Chem 269:25856-64).

In an embodiment of the invention, splicing enhancers such as, for example, sequences referred to as exonic splicing enhancers may also be included in the structure of the synthetic PTMs. Transacting splicing factors, namely the serine/arginine-rich (SR) proteins, have been shown to interact with such exonic splicing enhancers and modulate splicing (See, Tacke et al., 1999, Curr. Opin. Cell Biol. 11:358-362; Tian et al., 2001, J. Biological Chemistry 276:33833-33839; Fu, 1995, RNA 1:663-680). Nuclear localization signals may also be included in the PTM molecule (Dingwell and Laskey, 1986, Ann. Rev. Cell Biol. 2:367-390; Dingwell and Laskey, 1991, Trends in Biochem. Sci. 16:478-481). Such nuclear localization signals can be used to enhance the transport of synthetic PTMs into the nucleus where trans-splicing occurs.

Additional features can be added to the PTM molecule either after, or before, the nucleotide sequence encoding a translatable protein, such as polyadenylation signals to modify RNA expression/stability, or 5′ splice sequences to enhance splicing, additional binding regions, “safety”-self complementary regions, additional splice sites, or protective groups to modulate the stability of the molecule and prevent degradation. In addition, stop codons may be included in the PTM structure to prevent translation of unspliced PTMs. Further elements such as a 3′ hairpin structure, circularized RNA, nucleotide base modification, or synthetic analogs can be incorporated into PTMs to promote or facilitate nuclear localization and spliceosomal incorporation, and intra-cellular stability.

PTMs may also be generated that require a double-trans-splicing reaction for generation of a chimeric trans-spliced product. PTMs designed to promote two trans-splicing reactions are engineered as described above, however, they contain both 5′ donor sites and 3′ splice acceptor sites. In addition, the PTMs may comprise two or more binding domains and splice regions. The splice regions may be placed between the multiple binding domains and splice sites or alternatively between the multiple binding domains.

When specific PTMs are to be synthesized in vitro (synthetic PTMs), such PTMs can be modified at the base moiety, sugar moiety, or phosphate backbone, for example, to improve stability of the molecule, hybridization to the target mRNA, transport into the cell, etc. For example, modification of a PTM to reduce the overall charge can enhance the cellular uptake of the molecule. In addition modifications can be made to reduce susceptibility to nuclease or chemical degradation. The nucleic acid molecules may be synthesized in such a way as to be conjugated to another molecule such as a peptides (e.g., for targeting host cell receptors in vivo), or an agent facilitating transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents (see, e.g., Krol et al., 1988, Bio. Techniques 6:958-976) or intercalating agents (see, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life. Possible modifications include, but are not limited to, the addition of flanking sequences of ribonucleotides to the 5′ and/or 3′ ends of the molecule. In some circumstances where increased stability is desired, nucleic acids having modified internucleoside linkages such as 2′-O-methylation may be preferred. Nucleic acids containing modified internucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein).

The synthetic PTMs of the present invention are preferably modified in such a way as to increase their stability in the cells. Since RNA molecules are sensitive to cleavage by cellular ribonucleases, it may be preferable to use as the competitive inhibitor a chemically modified oligonucleotide (or combination of oligonucleotides) that mimics the action of the RNA binding sequence but is less sensitive to nuclease cleavage. In addition, the synthetic PTMs can be produced as nuclease resistant circular molecules with enhanced stability to prevent degradation by nucleases (Puttaraju et al., 1995, Nucleic Acids Symposium Series No. 33:49-51; Puttaraju et al., 1993, Nucleic Acid Research 21:4253-4258). Other modifications may also be required, for example to enhance binding, to enhance cellular uptake, to improve pharmacology or pharmacokinetics or to improve other pharmaceutically desirable characteristics.

Modifications, which may be made to the structure of the synthetic PTMs include but are not limited to backbone modifications such as use of:

(i) phosphorothioates (X or Y or W or Z═S or any combination of two or more with the remainder as O). e.g. Y═S (Stein, C. A., et al., 1988, Nucleic Acids Res., 16:3209-3221), X═S (Cosstick, R., et al., 1989, Tetrahedron Letters, 30, 4693-4696), Y and Z=S (Brill, W. K.-D., et al., 1989, J. Amer. Chem. Soc., 111:2321-2322); (ii) methylphosphonates (e.g. Z=methyl (Miller, P. S., et al., 1980, J. Biol. Chem., 255:9659-9665); (iii) phosphoramidates (Z═N-(alkyl)₂ e.g. alkyl methyl, ethyl, butyl) (Z=morpholine or piperazine) (Agrawal, S., et al., 1988, Proc. Natl. Acad. Sci. USA 85:7079-7083) (X or W═NH) (Mag, M., et al., 1988, Nucleic Acids Res., 16:3525-3543); (iv) phosphotriesters (Z═O-alkyl e.g. methyl, ethyl, etc) (Miller, P. S., et al., 1982, Biochemistry, 21:5468-5474); and (v) phosphorus-free linkages (e.g. carbamate, acetamidate, acetate) (Gait, M. J., et al., 1974, J. Chem. Soc. Perkin 1,1684-1686; Gait, M. J., et al., 1979, J. Chem. Soc. Perkin 1,1389-1394).

In addition, sugar modifications may be incorporated into the PTMs of the invention. Such modifications include the use of: (i) 2′-ribonucleosides (R═H); (ii) 2′-O-methylated nucleosides (R═OMe)) (Sproat, B. S., et al., 1989, Nucleic Acids Res., 17:3373-3386); and (iii) 2′-fluoro-2′-riboxynucleosides (R═F) (Krug, A., et al., 1989, Nucleosides and Nucleotides, 8:1473-1483).

Further, base modifications that may be made to the PTMs, including but not limited to use of: (i) pyrimidine derivatives substituted in the 5-position (e.g. methyl, bromo, fluoro etc) or replacing a carbonyl group by an amino group (Piccirilli, J. A., et al., 1990, Nature, 343:33-37); (ii) purine derivatives lacking specific nitrogen atoms (e.g. 7-deaza adenine, hypoxanthine) or functionalized in the 8-position (e.g. 8-azido adenine, 8-bromo adenine) (for a review see Jones, A. S., 1979, Int. J. Biolog. Macromolecules, 1:194-207).

In addition, the PTMs may be covalently linked to reactive functional groups, such as: (i) psoralens (Miller, P. S., et al., 1988, Nucleic Acids Res., Special Pub. No. 20, 113-114), phenanthrolines (Sun, J-S., et al., 1988, Biochemistry, 27:6039-6045), mustards (Vlassov, V. V., et al., 1988, Gene, 72:313-322) (irreversible cross-linking agents with or without the need for co-reagents); (ii) acridine (intercalating agents) (Helene, C., et al., 1985, Biochimie, 67:777-783); (iii) thiol derivatives (reversible disulphide formation with proteins) (Connolly, B. A., and Newman, P. C., 1989, Nucleic Acids Res., 17:4957-4974); (iv) aldehydes (Schiffs base formation); (v) azido, bromo groups (UV cross-linking); or (vi) ellipticines (photolytic cross-linking) (Perrouault, L., et al., 1990, Nature, 344:358-360).

In an embodiment of the invention, oligonucleotide mimetics in which the sugar and internucleoside linkage, i.e., the backbone of the nucleotide units, are replaced with novel groups can be used. For example, one such oligonucleotide mimetic which has been shown to bind with a higher affinity to DNA and RNA than natural oligonucleotides is referred to as a peptide nucleic acid (PNA) (for review see, Uhlmann, E. 1998, Biol. Chem. 379:1045-52). Thus, PNA may be incorporated into synthetic PTMs to increase their stability and/or binding affinity for the target pre-mRNA.

In another embodiment of the invention synthetic PTMs may be covalently linked to lipophilic groups or other reagents capable of improving uptake by cells. For example, the PTM molecules may be covalently linked to: (i) albumin (Osborn, B. et al., 2002 J. Pharmcol. Ther. 303:540-548) (ii) cholesterol (Letsinger, R. L., et al., 1989, Proc. Natl. Acad. Sci. USA, 86:6553-6556); (iii) polyamines (Lemaitre, M., et al., 1987, Proc. Natl. Acad. Sci, USA, 84:648-652); other soluble polymers (e.g. polyethylene glycol) to improve the efficiently with which the PTMs are delivered to a cell. In addition, combinations of the above identified modifications may be utilized to increase the stability and delivery of PTMs into the target cell. The PTMs of the invention can be used in methods designed to produce a novel chimeric RNA in a target cell.

The methods of the present invention comprise delivering to the target cell a PTM which may be in any form used by one skilled in the art, for example, an RNA molecule, or a DNA vector which is transcribed into a RNA molecule, wherein said PTM binds to a pre-mRNA and mediates a trans-splicing reaction resulting in formation of a chimeric RNA comprising a portion of the PTM molecule spliced to a portion of the pre-mRNA.

In a specific embodiment of the invention, the PTMs of the invention can be used in methods designed to produce a novel chimeric RNA in a target cell so as to result in expression of a fusion protein comprising an immunogenic peptide of interest and a polypeptide involved in antigen uptake, presentation and chaperoning. The methods of the present invention comprise delivering to a cell a PTM which may be in any form used by one skilled in the art, for example, an RNA molecule, or a DNA vector which is transcribed into a RNA molecule, wherein said PTM binds to a target pre-mRNA and mediates a trans-splicing reaction resulting in formation of a chimeric RNA comprising the portion of the PTM molecule spliced to a portion of the target pre-mRNA.

### 5.2. Synthesis of the Trans-Splicing Molecules

The nucleic acid molecules of the invention can be RNA or DNA or derivatives or modified versions thereof, single-stranded or double-stranded. By nucleic acid is meant a PTM molecule or a nucleic acid molecule encoding a PTM molecule, whether composed of deoxyribonucleotides or ribonucleosides, and whether composed of phosphodiester linkages or modified linkages. The term nucleic acid also specifically includes nucleic acids composed of bases other than the five biologically occurring bases (adenine, guanine, thymine, cytosine and uracil). In addition, the PTMs of the invention may comprise, DNA/RNA, RNA/protein or DNA/RNA/protein chimeric molecules that are designed to enhance the stability of the PTMs.

The PTMs of the invention can be prepared by any method known in the art for the synthesis of nucleic acid molecules. For example, the nucleic acids may be chemically synthesized using commercially available reagents and synthesizers by methods that are well known in the art (see, e.g., Gait, 1985, Oligonucleotide Synthesis: A Practical Approach, IRL Press, Oxford, England).

Alternatively, synthetic PTMs can be generated by in vitro transcription of DNA sequences encoding the PTM of interest. Such DNA sequences can be incorporated into a wide variety of vectors downstream from suitable RNA polymerase promoters such as the T7, SP6, or T3 polymerase promoters. Consensus RNA polymerase promoter sequences include the following:

| | |
|---|---|
| T7: | TAATACGACTCACTATAGGGAGA |
| SP6: | ATTTAGGTGACACTATAGAAGNG |
| T3: | AATTAACCCTCACTAAAGGGAGA. |

The base in bold is the first base incorporated into RNA during transcription. The underline indicates the minimum sequence required for efficient transcription.

RNAs may be produced in high yield via in vitro transcription using plasmids such as SPS65 and Bluescript (Promega Corporation, Madison, Wis.). In addition, RNA amplification methods such as Q-β amplification can be utilized to produce the PTM of interest.

The PTMs may be purified by any suitable means, as are well known in the art. For example, the PTMs can be purified by gel filtration, affinity or antibody interactions, reverse phase chromatography or gel electrophoresis. Of course, the skilled artisan will recognize that the method of purification will depend in part on the size, charge and shape of the nucleic acid to be purified.

The PTM's of the invention, whether synthesized chemically, in vitro, or in vivo, can be synthesized in the presence of modified or substituted nucleotides to increase stability, uptake or binding of the PTM to a target pre-mRNA. In addition, following synthesis of the PTM, the PTMs may be modified with peptides, chemical agents, antibodies, or nucleic acid molecules, for example, to enhance the physical properties of the PTM molecules. Such modifications are well known to those of skill in the art.

In instances where a nucleic acid molecule encoding a PTM is utilized, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The DNA encoding the PTM of interest may be recombinantly engineered into a variety of host vector systems that also provide for replication of the DNA in large scale and contain the necessary elements for directing the transcription of the PTM. The use of such a construct to transfect target cells in the patient will result in the transcription of sufficient amounts of PTMs that will form complementary base pairs with the endogenously expressed pre-mRNA targets, and thereby facilitate a trans-splicing reaction between the complexed nucleic acid molecules. For example, a vector can be introduced in vivo such that is taken up by a cell and directs the transcription of the PTM molecule. Such a vector can remain episomal or become chromosomally integrated, as long as it can be transcribed to produce the desired RNA, i.e., PTM. Such vectors can be constructed by recombinant DNA technology methods standard in the art.

Vectors encoding the PTM of interest can be plasmid, viral, or others known in the art, used for replication and expression in mammalian cells. Expression of the sequence encoding the PTM can be regulated by any promoter/enhancer sequences known in the art to act in mammalian, preferably human cells. Such promoters/enhancers can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:14411445), the regulatory sequences of the metallothionein gene (Brinster et al., 1982, Nature 296:39-42), the viral CMV promoter, the human chorionic gonadotropin-β promoter (Hollenberg et al., 1994, Mol. Cell. Endocrinology 106:111-119), etc.

Any type of plasmid, cosmid, YAC or viral vector can be used to prepare the recombinant DNA construct which can be introduced directly into the tissue site. Alternatively, viral vectors can be used which selectively infect the desired target cell. This includes plasmids that have few, if any, bacterial sequences through a depletion of the bacterial backbone, such plasmids known generally as minicircle plasmids of the type described by Darquet et al (Gene Ther 6:209-218, 1999) and by Chen et al (Molecular Ther 8:495-500, 2003). Vectors for use in the practice of the invention include any eukaryotic expression vectors, including but not limited to viral expression vectors such as those derived from the class of retroviruses, adenoviruses or adeno-associated viruses.

A number of selection systems can also be used, including but not limited to selection for expression of the herpes simplex virus thymidine kinase, hypoxanthine-guanine phosphoribosyltransterase and adenine phosphoribosyl transferase protein in tk-, hgprt- or aprt-deficient cells, respectively. Also, anti-metabolic resistance can be used as the basis of selection for dihydrofolate transferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin. In a preferred embodiment of the invention, the cell culture is transformed at a low ratio of vector to cell such that there will be only a single vector, or a limited number of vectors, present in any one cell.

### 5.3. Uses and Administration of Trans-Splicing Molecules

The compositions and methods of the present invention may be used for induction of a protective immune response in a host. Specifically, targeted trans-splicing, including double-trans-splicing reactions, 3′ exon replacement and/or 5′ exon replacement can be used to form a chimeric RNA between the target pre-RNA and the PTM wherein said chimeric RNA encodes a fusion protein comprising the antigenic polypeptide of interest fused to a polypeptide involved in antigen uptake, presentation and chaperoning.

As used herein, the phrase “induction of a protective immune response”, and the like, is used broadly to include the induction of any immune-based response in a host, including either an antibody or cell-mediated immune response, or both, that serves to protect the host against the particular pathogen or cancer cell. Induction of a protective immune response also includes the induction of an autoimmune response against tissue-specific self antigens (Pardoll, D. M. 1999, PNAS 96:5340-5342). The term refers not only to the absolute prevention of any of the symptoms or conditions in the host resulting from infection with the particular pathogen, or from the cancer, but also to any detectable delay in the onset of any such symptoms or conditions, any detectable reduction in the degree or rate of infection by the particular pathogen, or any detectable reduction in the severity of the disease or any symptom or condition resulting from the presence of cancer cells, or from cells that are expressing autoimmune disease or allergy. Compositions according to the present invention, which comprise the antigenic polypeptide of interest, should be administered at a dosage and for a duration sufficient to reduce one or more clinical signs associated with the infection of the host.

Various delivery systems are known and can be used to transfer the compositions of the invention into cells, e.g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral, adenoviral, adeno-associated viral or other vector, injection of DNA, electroporation, calcium phosphate mediated transfection, etc. In a specific embodiment of the invention, albumin which has been shown to increase the half life of therapeutic molecules in the circulation may be associated with the PTMs, or nucleic acid molecules encoding the PTMS (Osborn, B. et al, 2002. J. Pharmacol. Exp. Ther. 303:540-548).

The compositions and methods can be used to provide a nucleic acid encoding an antigenic polypeptide to cells of an individual where expression of said polypeptide results in stimulation of a protective immune. Specifically, the compositions and methods can be used to provide sequences encoding an antigenic polypeptide of interest fused to a protein capable of enhancing immunity to cells of an individual to induce a protective immune response.

In a preferred embodiment, PTMS, or nucleic acids encoding a PTM are administered to promote PTM function, by way of gene delivery and expression into a host cell. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Delivery of the PTM into a host cell may be either direct, in which case the host is directly exposed to the PTM, or PTM encoding nucleic acid molecule, or indirect, in which case, host cells are first transformed with the PTM, or PTM encoding nucleic acid molecule in vitro, then transplanted into the host. These host cells can include tumor cells, lymphocytes, bone marrow cells, leukocytes, or antigen presenting cells such as dendritic cells and Langerhans cells. The administration could also be achieved ex vivo where cells of the host are removed and the PTM is applied in a variety of formulations or other cells which can stimulate the induction of the desired immune response. These two approaches are known, respectively, as in vivo or ex vivo gene delivery.

In a specific embodiment, the nucleic acid is directly administered in vivo. This can be accomplished by any of numerous methods known in the art, e.g., by constructing it as part of an appropriate nucleic acid expression vector and administering it so that it becomes intracellular, e.g. by infection using a defective or attenuated retroviral or other viral vector (see U.S. Pat. No. 4,980,286), or by direct injection of naked DNA, or by use of microparticle bombardment (e.g., a gene gun; Biolistic, Dupont, Bio-Rad), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432), or by delivery via a bronchoscope, endoscope or other means known in the art.

In a specific embodiment, a viral vector that contains the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In a preferred embodiment of the invention an adeno-associated viral vector may be used to deliver nucleic acid molecules capable of encoding the PTM. The vector is designed so that, depending on the level of expression desired, the promoter and/or enhancer element of choice may be inserted into the vector.

In yet another embodiment of the invention, PTMs, or nucleic acid molecules encoding said PTM, are administered ex vivo. Such an approach involves transferring the PTM, or molecule encoding a PTM, to cells in tissue culture by such methods as electroporation, lipofection, calcium phosphate mediated transfection, or viral infection. The method of transfer may further include the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host's cell.

Additionally, cells comprising the PTMs of the invention may be further engineered to express cytokine/growth factors that can facilitate the recruitment of immunologic cells to the cell comprising the PTM. Such cytokine/growth factors are well know to those of skill in the art and include, for example, granulocyte macrophage stimulating cell growth factor (GMCSF), interleukins or similarly acting molecules.

The present invention relates to the use of the nucleic acid molecules of the invention as vaccines. In an embodiment of the invention, such nucleic acids may be directly injected into the host, or cells of the host (see, Wolff, J. 1990, Science 247:1465-1468; Tang, D. et al., 1992, Nature 356:152-154; Ulmer, J. B. et al., 1993, Science 259:1745-1749; Huygen, 2003 Infect Immun 71:1613-1621; Haupt, K, et al., Exper. Biol. Med 227-227-237; Wolff, 1992, Hum. Mol. Genet. 1:363-369). In a specific embodiment of the invention, the vaccine may be targeted to DCs (You, Z. et al., 2001, Cancer Res 61:3704-3711, 2001; Deliyannis, G. et al. 2000, PNAS. 97:6676-6680).

It is expected that, as a result of nucleic acid vaccination, both cellular (CD4+ and CD8+ T cells) and humoral responses can be induced because the encoded antigen is processed in both the endogenous and exogenous pathways. Thus, resulting peptide epitopes are presented by major histocompatibility complexes (MHC) I and II.

The present invention also provides for pharmaceutical compositions comprising an effective amount of a PTM or a nucleic acid encoding a PTM, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical Sciences” by E. W. Martin.

In specific embodiments, pharmaceutical compositions are administered in diseases or disorders involving infection with a pathogenic microorganism. Such pathogens include, viruses, bacteria, fungi and parasites. The pharmaceutical compositions of the invention may be administer to prevent, or to treat, diseases or disorders arising from infection with a pathogenic microorganism.

In another specific embodiment of the invention, pharmaceutical compositions are administered in proliferative diseases or disorders, such as cancer. The pharmaceutical compositions are designed to induce an immune response against antigens that are either tumor-specific or tumor-associated with the objective of inducing an immunologic response to cancer, resulting either in prophylactic protection against tumor challenge, or as a therapeutic vaccine against the cancer. Alternately, the pharmaceutical compositions are designed to induce an autoimmune response against tissue specific self antigens that will be important as a therapeutic against diseases such as cancer.

A variety of different administration techniques may be used to administer the compositions of the invention, including intradermal, subcutaneous, intratumoral, intra-lymphatic or intramuscular inoculations, or such procedures that would allow the PTM to be incorporated into the proper cell for processing.

The PTM will be administered in amounts which are effective to produce the desired effect in the targeted cell. Effective dosages of the PTMs can be determined through procedures well known to those in the art which address such parameters as biological half-life, bioavailability and toxicity. The amount of the composition of the invention which will be effective will depend on the disease or disorder being treated, and can be determined by standard clinical techniques. Such techniques include analysis of blood samples to determine the level of immunity induced. In addition, in vitro assays may optionally be employed to help identify optimal dosage ranges.

The present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human administration.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A cell comprising a nucleic acid molecule wherein said nucleic acid molecule comprises:
a) one or more target binding domains that target binding of the nucleic acid molecule to a target pre-mRNA expressed within the cell, wherein said target pre-mRNA encodes a protein involved in antigen uptake, presentation or chaperoning;
b) a splice region;
c) a spacer region that separates the splice region from the target binding domain; and
d) a nucleotide sequence to be trans-spliced to the target pre-mRNA wherein said nucleotide sequence encodes an antigen of interest;
wherein said nucleic acid molecule is recognized by nuclear splicing components within the cell.

2. The cell of claim 1 wherein the splice region comprises a 3′ splice region.

3. The cell of claim 1 wherein the splice region comprises a 5′ splice region.

4. The cell of claim 2 wherein the 3′ splice region comprises at least one of a branch point and a 3′ splice acceptor site.

5. The cell of claim 2 wherein the 3′ splice region further comprises a pyrimidine tract.

6. The cell of claim 2 wherein the nucleic acid molecule further comprises a safety nucleotide sequence comprising one or more complementary sequences that bind to one or more sides of the 3′ splice region

7. The cell of claim 3 wherein said nucleic acid molecule further comprises a safety sequence comprising one or more complementary sequences that bind to one or both sides of the 5′ splice site.

8. The cell of claim 3 wherein the nucleic acid molecule further comprises a 5′ donor site.

9. A method of producing a chimeric RNA molecule in a cell comprising:
contacting a target pre-mRNA expressed in the cell with a nucleic acid molecule recognized by nuclear splicing components wherein said nucleic acid molecule comprises:
a) one or more target binding domains that target binding of the nucleic acid molecule to a target pre-mRNA expressed within the cell, wherein said target pre-mRNA encodes an protein involved in antigen uptake, presentation or chaperoning;
b) a splice region;
c) a spacer region that separates the splice region from the target binding domain; and
d) a nucleotide sequence to be trans-spliced to the target pre-mRNA wherein said nucleotide sequence encodes an antigen of interest;
under conditions in which a portion of the nucleic acid molecule is trans-spliced to a portion of the target pre-mRNA to form a chimeric RNA within the cell.

10. The method of claim 9 wherein the splice region comprises a 3′ splice region.

11. The method of claim 9 wherein the splice region comprises a 5′ splice region.

12. The method of claim 10 wherein the 3′ splice region comprises at least one of a branch point and a 3′ splice acceptor site.

13. The method of claim 10 wherein the 3′ splice region further comprises a pyrimidine tract.

14. The method of claim 10 wherein the nucleic acid molecule further comprises a safety nucleotide sequence comprising one or more complementary sequences that bind to one or more sides of the 3′ splice region

15. The method of claim 11 wherein said nucleic acid molecule further comprises a safety sequence comprising one or more complementary sequences that bind to one or both sides of the 5′ splice site.

16. The method of claim 11 wherein the nucleic acid molecule further comprises a 5′ donor site.

17. A nucleic acid molecule comprising:
a) one or more target binding domains that target binding of the nucleic acid molecule to a target pre-mRNA expressed within the cell, wherein said target pre-mRNA encodes an protein involved in antigen uptake, presentation or chaperoning;
b) a splice region;
c) a spacer region that separates the splice region from the target binding domain; and
d) a nucleotide sequence to be trans-spliced to the target pre-mRNA wherein said nucleotide sequence encodes an antigen of interest;
wherein said nucleic acid molecule is recognized by nuclear splicing components within the cell.

18. The nucleic acid of claim 17 wherein the splice region comprises a 3′ splice region.

19. The nucleic acid of claim 17 wherein the splice region comprises a 5′ splice region.

20. The nucleic acid of claim 18 wherein the 3′ splice region comprises at least one of a branch point and a 3′ splice acceptor site.

21. The nucleic acid of claim 18 wherein the 3′ splice region further comprises a pyrimidine tract.

22. The nucleic acid of claim 18 wherein the nucleic acid molecule further comprises a safety nucleotide sequence comprising one or more complementary sequences that bind to one or more sides of the 3′ splice region

23. The nucleic acid of claim 19 wherein said nucleic acid molecule further comprises a safety sequence comprising one or more complementary sequences that bind to one or both sides of the 5′ splice site.

24. The nucleic acid of claim 19 wherein the nucleic acid molecule further comprises a 5′ donor site.

25. A nucleic acid molecule comprising:
a) a splice region;
b) a spacer region that separates the splice region from the target binding domain; and
c) a nucleotide sequence to be trans-spliced to the target pre-mRNA wherein said nucleotide sequence encodes an antigen of interest;
wherein the nucleic acid molecule targets a pre-mRNA expressed within the cell, wherein the pre-mRNA encodes a protein involved in antigen uptake, presentation or chaperoning and wherein said nucleic acid molecule is recognized by nuclear splicing components within the cell.

26. The nucleic acid of claim 25 wherein the splice region comprises a 3′ splice region.

27. The nucleic acid of claim 25 wherein the splice region comprises a 5′ splice region.

28. The nucleic acid of claim 26 wherein the 3′ splice region comprises at least one of a branch point and a 3′ splice acceptor site.

29. The nucleic acid of claim 26 wherein the 3′ splice region further comprises a pyrimidine tract.

30. The nucleic acid of claim 26 wherein the nucleic acid molecule further comprises a safety nucleotide sequence comprising one or more complementary sequences that bind to one or more sides of the 3′ splice region

31. The nucleic acid of claim 27 wherein said nucleic acid molecule further comprises a safety sequence comprising one or more complementary sequences that bind to one or both sides of the 5′ splice site.

32. The nucleic acid of claim 27 wherein the nucleic acid molecule further comprises a 5′ donor site.


SEQUENCE LISTING (6 sequences)

SEQ ID NO: 1 - DNA, 258 bp, Unknown
  unable to identify
gtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttccacatctttatattttgcaccacattcaacactgtagcggccgc

SEQ ID NO: 2 - DNA, 270 bp, Unknown
  unable to identify
ccaactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccag

SEQ ID NO: 3 - RNA, 18 nt, Unknown
  unable to identify
gggcugauuuuuccaugu

SEQ ID NO: 4 - DNA, 23 bp, Unknown
  unable to identify
taatacgactcactatagggaga

SEQ ID NO: 5 - DNA, 23 bp, Unknown
  unable to identify
atttaggtgacactatagaagng

SEQ ID NO: 6 - DNA, 23 bp, Unknown
  unable to identify
aattaaccctcactaaagggaga